(19) United States
(12) Patent Application Publication
(10) Pub. No.: US 2014/0257710 A1
(43) Pub. Date: Sep. 11, 2014

(54) METHOD AND SYSTEM FOR ANALYZING THE TAXONOMIC COMPOSITION OF A METAGENOME IN A SAMPLE

(71) Applicant: OFEK ESHKOLOT RESEARCH AND DEVELOPMENT LTD., (US)

(72) Inventors: Vladimir VOLKOVICH, Karmiel (IL); Valery KIRZHNER, Haifa (IL)

(73) Assignee: OFEK ESHKOLOT RESEARCH AND DEVELOPMENT LTD., Karmiel (IL)

(21) Appl. No.: 13/787,861

(22) Filed: Mar. 7, 2013

(51) Int. Cl. G06F 19/22; C12Q 1/68

(52) U.S. Cl. CPC G06F 19/22; C12Q 1/689
USPC 702/20; 435/6.1; 435/6.15

(57) ABSTRACT

Provided herein are methods and systems for rapid identification and quantification of the taxonomic composition of a microbial metagenome in a sample, based on compositional spectra analysis. The methods and systems are useful in diagnostic and analytic methods in the clinic and in the field.

### FIELD OF THE INVENTION

Provided herein are a method and a system for rapid identification and quantification of the taxonomic composition of a microbial metagenome in a sample, based on the compositional spectra analysis.

### BACKGROUND OF THE INVENTION

Currently used methods for the detection of microbes, for example, pathenogenic or environmentally detrimental bacteria in clinical or environmental samples rely primarily on PCR, which is based on identifying the presence of a unique DNA sequence in a mixture of DNA and requires primers to multiple microbial genomes. Other methods include DNA arrays and radiolabel or fluorescent detection. Kirzhner et al. describe genomic sequencing characterization and comparison based on the compositional spectra (CS) of short DNA sequences (Physica A 312 (2002) 447-57).

The recently developed metagenomic approach, allows analysis of microorganisms at a different level. A metagenome is the entire set of bacterial genomes in an organism, in a sample or, for example, in an organ as the intestines. Identifying the presence and composition of the microorganism communities in a sample has broad use in the clinic, in industry and in the field. For example, in humans, the metagenome is dynamic because the corresponding community of microorganisms is under the continuous influence of changing factors such as nutrition and medicine. A mathematical method intended to solve such problems was recently proposed (Meinicke, et al., Bioinformatics, 2011, 27 (12):1618-1624). This method appears to be effective for quantifying bacteria when the metagenome content is known and it is only necessary to follow the concentrations of bacteria. In this case, the computational time is as short as several seconds or minutes. Meinicke et al. does not take into consideration circumstances in which one or more of the genomes in a metagenome is unknown or two or more genomes have similar spectra (for example are evolutionarily related).

The methods known in the art are deficient in that they inaccurately quantify the microbes and the relative ratio of each genome in a mixture of genomes. A method for the accurate identification and quantification of variable populations of microorganisms is desired for diagnostics, monitoring treatment and epidemiological analyses. For example, precise knowledge of the metagenome composition infecting a patient would allow targeted pharmacological therapy of the patient thereby reducing complications, side effects and development of antibiotic resistance. There remains a need for a system and method for rapid and accurate analysis of dynamic metagenomes where the taxonomic composition is known or partially known.

### SUMMARY OF THE INVENTION

Provided herein are a method and a system for rapid identification and quantification of the taxonomic composition of a microbial metagenome in a sample. The method and system are based on the fact that the statistical distribution of the fixed-length strings of nucleotides (words) over the whole genome (compositional spectrum) is specific for each genome. The output of the sequenator is a set of fixed-length words, associated with a genome, which is a component of the metagenome under study. Without wishing to be bound to theory, a sequenator generates a mixture of compositional spectra of all the genomes comprising the metagenome, with account for their multiplicity. The algorithm disclosed herein separates the compositional spectra mixture using the compositional spectra of known genomes.

In one aspect, provided herein is a method for characterizing a microorganism metagenome in a sample, the method comprising
a) providing a compositional spectra mixture from genomic sequences of genomes comprising the microorganism metagenome in the sample;
b) providing a compositional spectra set of known microorganism genomic sequences,
c) characterizing sequences in the compositional spectra mixture of (a) using the compositional spectra set of (b), wherein said characterizing
   comprises solving a linear system by (i) providing a vector of representations of said sequences in said compositional spectra mixture of (a); and (ii) comparing representations in said vector to representations of sequences in said compositional spectra set of (b).

In some embodiments, step (c) is performed by a suitably configured processor of a computer system stored on a computer readable medium configured to receive the compositional spectra mixture and the compositional spectra set. In alternate embodiments, step (c) is performed by a suitably configured processor of a computer system stored on a computer readable medium comprising the database of known microorganism genomic sequences, and configured to receive the compositional spectra mixture.

In some embodiments, the compositional spectra set of known microorganism genomic sequences is obtained from a publicly available database. In other embodiments the compositional spectra set of known microorganism genomic sequences is obtained from a subset of a publicly available database.

In some embodiments, the providing said compositional spectra mixture in step (a) comprises employing a sequenator to provide said compositional spectra mixture.

In various embodiments, providing the compositional spectra mixture comprises providing fixed length strings of nucleosides (words) based on the genomic sequences. The fixed-length string of nucleosides is 4 to 20 nucleotides in length, or 6 to 10 nucleotides in length, or preferably 6 nucleotides in length.

In some embodiments, each genome sequence is composed of sequence segments of 10 to 10,000 nucleotides in length, or 100 to 1,000 nucleotides in length.

In some embodiments, the metagenome in the sample consists of genome of a single microorganism. In other embodiments, the metagenome in the sample consists of a plurality of microorganisms.

In some embodiments, the characterizing comprises identifying and quantifying each microorganism genome of the metagenome in the sample.

In some embodiments, the method further comprises the addition of a microorganism genome having a known genomic sequence to the sample prior to providing the genomic sequence. Preferably the added genome is unrelated to the metagenome of the sample.

In some embodiments of the method, the sample is a food or beverage sample; a human/animal sample (contents of stomach or intestine; urine; blood; vaginal secretion; fecal matter, phlegm (sputum), cerebrospinal fluid (CSF), pus, synovial fluid) or an environmental specimen (water, plant material or soil).

In some embodiments of the method, the genomic sequence is obtained from a standard sequenator. In some embodiments the sequenator output comprises whole genomic sequence. In some embodiments the sequenator output comprises the compositional spectrum of a single genome.

In some embodiments of the method, the sample comprises a microorganism, which is a bacterium (including a mycoplasma), a virus, a protozoa or a spore. In some embodiments of the method, the sample comprises a plurality of microorganisms, which are bacteria, viruses, protozoa, spores or a combination of such microorganisms. The terms “microorganism” and a “microbe” are used interchangeably herein.

In another aspect, provided herein is a microbial metagenome analyzing system. The system comprises a computer means configured to:
generate compositional spectra set of known genome sequences;
form a stable system matrix by preprocessing a linear system derived from the compositional spectra;
characterize a compositional spectra mixture of the metagenome of the sample using the stable system matrix to solve the linear system by (i) providing a vector of the compositional spectra mixture of the sample's metagenome; and (ii) comparing the vector values to the compositional spectra set of the known microorganism genomes.

The characterization step is performed by a suitably configured processor of a computer system stored on a computer readable medium configured to receive the compositional spectra mixture and the compositional spectra set. Alternatively, the characterization step is performed by a suitably configured processor of a computer system stored on a computer readable medium comprising the database of known microorganism genomic sequences, and configured to receive the compositional spectra mixture.

In another aspect, provided is a machine-readable storage medium comprising a program containing a set of instructions for causing a microbial metagenome analyzing system to execute procedures for determining the identity and multiplicity of the microbial metagenome in a sample. The machine readable storage medium comprises a program containing a set of instructions for causing a system to execute procedures for characterizing the metagenome in the sample, the procedures comprising:
generating a compositional spectra set of known genome sequences;
forming a stable system matrix by preprocessing a linear system derived from the compositional spectra;
characterizing a compositional spectra mixture of the metagenome of the sample using the stable system matrix to solve the linear system by (i) providing a vector of representations of said sequences in said compositional spectra set; and (ii) comparing representations in said vector to representations of sequences in said compositional spectra mixture.

In one embodiment, the machine-readable storage medium comprises programs consisting of a set of instructions for causing a microbial metagenome analyzing system to execute procedures set forth in FIG. 16A. In a preferred embodiment, the machine readable storage medium comprises programs consisting of a set of instructions for causing a microbial metagenome analyzing system to execute procedures set forth in FIG. 16B.

After the characterization in completed, images and data can be reviewed with the system's image review, data review, and summary review facilities. All images, data and settings can be archived in the system's database for later review or for interfacing with a network information management system. Data can also be exported to other third-party packages to tabulate results and generate reports. Data is reviewed and or analyzed by a user by implementing a combination of interactive graphs, data spreadsheets of measured features, and images. Graphical capabilities are further provided in which data can be viewed and or analyzed via interactive graphs such as histograms and scatter plots. Hard copies of data, images, graphs and the like can be printed on a wide range of standard printers. Finally, reports can be generated for example, users can generate a graphical report of data summarized on a sample-by-sample basis. This report includes a summary of the statistics by well in tabular and graphical format and identification information on the sample. The report window allows the operator to enter comments about the scan for later retrieval. Multiple reports can be generated on many statistics and be printed with the touch of one button. Reports can be previewed for placement and data before being printed. Such report are used, for example, by a physician, diagnostician or pathologist to assess efficacy of therapeutic treatment over time; by epidemiologists to trace origin or migration of diseases; or by field analysts to trace presence of pathogens in environmental samples and their migration or waning following treatment.

The methods, materials, systems and examples that will now be described are illustrative only and are not intended to be limiting; materials and methods similar or equivalent to those described herein can be used in practice or testing of the invention. Other features and advantages of the invention will be apparent from the following detailed description, and from the claims.

This disclosure is intended to cover any and all adaptations or variations of combination of features that are disclosed in the various embodiments herein. Although specific embodiments have been illustrated and described herein, it should be appreciated that the invention encompasses any arrangement of the features of these embodiments to achieve the same purpose. Combinations of the above features, to form embodiments not specifically described herein, will be apparent to those of skill in the art upon reviewing the instant description.

### BRIEF DESCRIPTION OF THE FIGURES

FIG. 1 shows a graph of the distribution of the cosines values for the angles between all possible compositional spectra (CS) pairs for approximately 1300 bacterial genomes. X-axis: cosines values ×100; Y-axis: the number of cosine values.

FIG. 2 shows a table with a set of 100 Eubacteria genomes, which represent all the main groups of bacteria. The number of genomes in each group is approximately proportional to the number of sequenced genomes in each group. The choice of genomes within the groups is random.

FIG. 3 shows a set of 28 bacteria genomes, which are characterized in Qin et al. (Nature (2007) 464:59-65) as the most common gut bacteria.

FIG. 4 is a table which presents the results of the calculations of the genome multiplicities in the mixture of 100 genomes for different segment lengths for the deterministic case. N refers to genome number from table in FIG. 2; OM refers to original multiplicity; 10, 20, . . . , 10000—segment lengths in the mixture; the last column—mixture of whole genomes.

FIG. 5 shows the distribution of the cosines of the angles between all possible vector pairs. The number of genomes in the sets: (a) 100; (b) 28.

FIG. 6 shows a table with the results of the calculations of the genome multiplicities in the mixture of 28 genomes for different segment lengths for the deterministic case. N refers to genome number in Table in FIG. 3; OM refers to original multiplicity; 10, 20, . . . , 10000—segment lengths in the mixture; the last column—mixture of whole genomes.

FIG. 7 shows a graph of the mean differences between the calculated and the actual genome multiplicities in the mixture as a function of the segment length (log scale is used for the x-axis) for sets M₁₀₀ and M₂₈. The mixture is composed of: (1) the whole set M₁₀₀ and the separating matrix contains all the genomes; (2) the whole set M₁₀₀, but the separating matrix contains only one genome of each almost collinear pair. The mean differences are obtained based on the difference between the calculated (non-integer) and the actual multiplicity; (3) the same as in (2), but the obtained multiplicity is approximated to the nearest integer; (4) the whole set M₂₈ and the separating matrix contains all the genomes.

FIG. 8 is a histogram of the expansion coefficients for the set of 11 E. coli genomes over the set of 100 genomes, one of these being also an E. coli genome.

FIG. 9 shows a table, with the results of the calculations of the genome multiplicities in the mixture of 28 genomes for different segment lengths for the deterministic case. N represents the genome number in the table in FIG. 2; OM represents the original multiplicity; 10, 20, . . . , 10000—segment lengths in the mixture; the last column—mixture of whole genomes.

FIG. 10 shows a table with the mean multiplicity (d) and the squared deviation (σ) for each bacterium of set M₁₀₀. Averaging is performed over 100 experiments in each series. N represents the genome number in the table of FIG. 2; OM represents the original multiplicity; 10, 20, . . . , 10000—segment lengths in the mixture. All the values are normalized by the 1^{st} genome on the list.

FIG. 11 is a graph which shows the dependence of the mean error in evaluating the genome multiplicities in a mixture on the segment length (log scale is used for the x-axis) for genome sets M₁₀₀ (circles) and M₂₈ (squares).

FIG. 12 is a graph, which shows the dependence of the mean-squared deviation of the genome multiplicities in a mixture on the segment length (log scale is used for the x-axis) for genome sets M₁₀₀ (circles) and M₂₈ (squares).

FIG. 13 is a bar graph depicting the actual (1) and the calculated multiplicities for each genome from set M₂₈ at C=50 (2) and C=10000 (3).

FIG. 14 provides a graph with the dynamics of the angles between the new and the earlier sets of genomes over the last ten years. X-axis: years. Y-axis: cosine values of the angles between CS of genomes. For each genome sequenced in a particular year, the minimal angle between this genome CS and CS of the genomes sequences up to this year is determined. The mean values of these angles cosines constitute the upper curve (squares). Each year, there appears a new genome which deviates from those already sequenced to the maximal extent, i.e. the one that has the greatest minimal angle. The lower curve (triangles) shows the cosines of these angles.

FIG. 15 presents a bar graph of (1) actual multiplicities; and (2) multiplicities calculated based on the 10-letter vocabulary (200 words with 3 mismatches) for a mixture of nine genomes: 1—Campylobacter1 jejuni; 2—Salmonella; 3—Pseudomonas aeruginosa; 4—Vibrio cholerae; 5—Mycobacterium tuberculosis; 6—Escherichia coli; 7—Legionella pneumophila; 8—Shigella boydii; 9—Yersinia enterocolitica.

FIGS. 16A and 16B provide flow charts showing methods of analyzing the microorganism metagenome in a sample.

### DETAILED DESCRIPTION OF THE INVENTION

The present method and system allow rapid and accurate identification and quantification of microorganisms in a sample and is applicable in a variety of settings, including clinical (i.e. diagnosis, treatment, detection of resistant bacteria); environmental (i.e. detection of toxic microorganisms in water, soil samples), industrial (i.e. identification of desirable or contaminating microorganisms in food and beverage products) forensic and defense (i.e. detection of biological warfare agents) and the like. Furthermore, provided is a clinically feasible method of monitoring treatment efficacy in a patient, by characterizing the metagenome in a patient and repeating metagenome characterization following treatment.

In one aspect, provided herein is a method for characterizing a microorganism metagenome in a sample, the method comprising
a) providing a compositional spectra mixture from genomic sequences of genomes comprising the microorganism metagenome in the sample;
b) providing a compositional spectra set of known microorganism genomic sequences,
c) characterizing sequences in the compositional spectra mixture of (a) using the compositional spectra set of (b), wherein said characterizing comprises solving a linear system by (i) providing a vector of representations of said sequences in said compositional spectra mixture of (a); and (ii) comparing representations in said vector to representations of sequences in said compositional spectra set of (b).

In some embodiments, step (c) is performed by a suitably configured processor of a computer system stored on a computer readable medium configured to receive the compositional spectra mixture and the compositional spectra set. In alternate embodiments, step (c) is performed by a suitably configured processor of a computer system stored on a computer readable medium comprising the database of known microorganism genomic sequences, and configured to receive the compositional spectra mixture.

In some embodiments, the compositional spectra set of known microorganism genomic sequences is obtained from a publicly available database. In other embodiments the compositional spectra set of known microorganism genomic sequences is obtained from a subset of a publicly available database. The compositional spectra mixture in step (a) may be obtained by employing a sequenator to provide said compositional spectra mixture.

In various embodiments, providing the compositional spectra mixture comprises providing fixed length strings of nucleosides (words) based on the genomic sequences. The fixed-length string of nucleosides is 4 to 20 nucleotides in length, 6 to 10 nucleotides in length, or preferably 6 nucleotides in length. In some embodiments, each genome sequence is composed of sequence segments of 10 to 10,000 nucleotides in length, or 100 to 1,000 nucleotides in length.

In some embodiments, the metagenome in the sample consists of genome of a single microorganism. In other embodiments, the metagenome in the sample consists of a plurality of microorganisms.

In some embodiments, the characterizing comprises identifying and quantifying each microorganism genome of the metagenome in the sample.

In some embodiments, the method further comprises the addition of a microorganism genome having a known genomic sequence to the sample prior to providing the genomic sequence. Preferably the added genome is unrelated to the metagenome of the sample.

In another aspect, provided herein is a microbial metagenome analyzing system. The system comprises a computer means configured to:
generate compositional spectra set of known genome sequences;
form a stable system matrix by preprocessing a linear system derived from the compositional spectra;
characterize a compositional spectra mixture of the metagenome of the sample using the stable system matrix to solve the linear system by (i) providing a vector of the compositional spectra mixture of the sample's metagenome; and (ii) comparing the vector values to the compositional spectra set of the known microorganism genomes.

The characterization step is performed by a suitably configured processor of a computer system stored on a computer readable medium configured to receive the compositional spectra mixture and the compositional spectra set. Alternatively, the characterization step is performed by a suitably configured processor of a computer system stored on a computer readable medium comprising the database of known microorganism genomic sequences, and configured to receive the compositional spectra mixture.

In another aspect, provided is a machine-readable storage medium comprising a program containing a set of instructions for causing a microbial metagenome analyzing system to execute procedures for determining the identity and multiplicity of the microbial metagenome in a sample. The machine readable storage medium comprises a program containing a set of instructions for causing a system to execute procedures for characterizing the metagenome in the sample, the procedures comprising:
generating a compositional spectra set of known genome sequences;
forming a stable system matrix by preprocessing a linear system derived from the compositional spectra;
characterizing a compositional spectra mixture of the metagenome of the sample using the stable system matrix to solve the linear system by (i) providing a vector of representations of said sequences in said compositional spectra set of the known genome sequences; and (ii) comparing representations in said vector to representations of sequences in said compositional spectra mixture from the genomes in the sample.

In some embodiments of the method, the system and medium, the sample is a food or beverage sample; a human/animal sample (contents of stomach or intestine; urine; blood; vaginal secretion; fecal matter, phlegm (sputum), cerebrospinal fluid (CSF), pus, synovial fluid) or an environmental specimen (water, plant material or soil).

In some embodiments of the method, the system and medium, the genomic sequence is obtained from a standard sequenator. In some embodiments the sequenator output comprises whole genomic sequence. In some embodiments the sequenator output comprises the compositional spectrum of a single genome.

In some embodiments of the method, the system and medium, the sample comprises a microorganism, the microorganism being a bacterium (including a mycoplasma), a virus, a protozoa or a spore. In some embodiments of the method, the sample comprises a plurality of microorganisms, which are bacteria, viruses, protozoa, spores or a combination of such microorganisms. The terms “microorganism” and a “microbe” are used interchangeably herein.

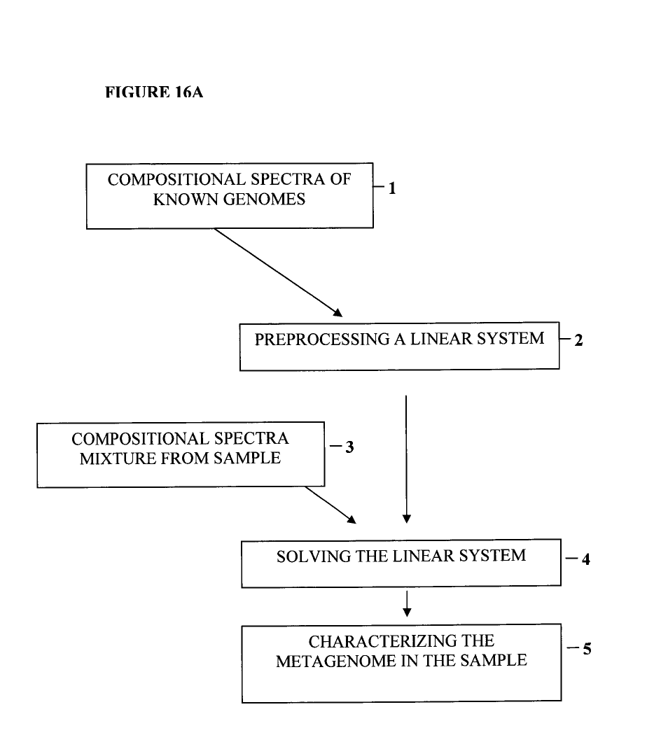

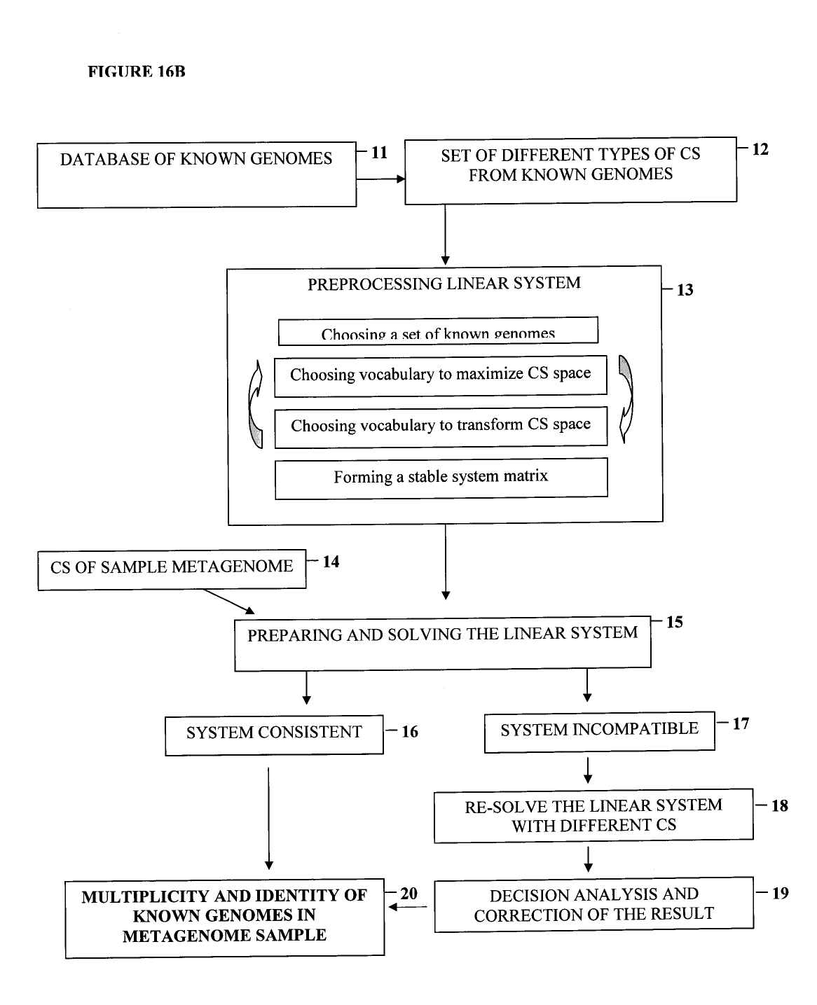

In one embodiment, the machine readable storage medium comprises programs consisting of a set of instructions for causing a microbial metagenome analyzing system to execute procedures set forth in FIG. 16A. In a preferred embodiment, the machine readable storage medium comprises programs consisting of a set of instructions for causing a microbial metagenome analyzing system to execute procedures set forth in FIG. 16B.

The following discussion describes the methods to characterize the metagenome in a sample illustrated in FIGS. 16A and 16B.

In FIG. 16A the primary steps of carrying out the method of characterizing the metagenome in a sample are provided: Compositional spectra of known microbial genomes is provided 1, based on genomic sequences of known microorganisms. The Cs may be obtained from public or private databases, or may be generated to fit the expected metagenome composition of the sample. A linear system (equation) is generated 2. The linear system is solved 4 by (i) providing a vector of the compositional spectra mixture of the sample's metagenome 3; and (ii) comparing the vector values to the compositional spectra set of the known microorganism genome sequences, thereby identifying the composition of the metagenome and the multiplicity of each genome in the metagenome in the sample 5. 4 is preferably performed with a suitably configured processor of a computer system stored on a computer readable medium configured to receive the compositional spectra mixture and the compositional spectra set.

In FIG. 16B, known microorganism genomes 11 is provided. A set of compositional spectra (CS) 12 is generated based on different set of words (oligonucleotide segments of different lengths.) The following steps carry out preprocessing of the linear system 13:
(i) choosing a set of known genomes for recognizing mixture
(ii) choosing a vocabulary to maximize CS space;
(iii) choosing a vocabulary for transforming CS space;
(iv) repeating the steps (ii) and (iii) until a stable system matrix is formed by excluding dependencies between the CS.

After the stable matrix of the known genomes is formed, the solution of the linear system 15 is calculated by separating the compositional spectra mixture 14 using the linear system of the compositional spectra set generated from 13.

If the system is consistent 16, then the identity and multiplicity of the genomes in the metagenome are provided 20. A consistent system is one in which all the genomes in the metagenome are represented in the database.

However, if the system is not compatible 17, then the identity and multiplicity of the genomes in the metagenome 20 are provided only after repeating the step of solving the linear system with a different CS 18, and analyzing and correcting the result 19.

In some embodiments of the method, the system and the medium, the sample is a food or beverage sample; a human/animal sample (contents of stomach or intestine; urine; blood, vaginal secretion; fecal matter, phlegm (sputum), cerebrospinal fluid (CSF) pus, synovial fluid) or an environmental specimen (water, plant material or soil).

In some embodiments of the method and the system, and the m medium, the compositional spectra mixture is generated from genomic sequences obtained from a standard sequenator. In some embodiments the sequenator output comprises a genome sequence, preferably whole genome sequence. Genomic sequencing may be performed by any of the methods known in the art, including but not limited to shotgun sequencing technology pure pairwise end sequencing automated capillary sequencers, pyrosequencing, or nanopore or fluorophore technology.

### Database

A database of known microorganism genomes may be obtained, for example, from the NCBI (National Center for Biotechnology Information), the European Bioinformatics Institute (EBI) and/or the DNA Data Bank of Japan (DDBJ) where they are stored as tests of the alphabet {A,T,C,G}. A database may also be generated from a limited number of genome sequences. In a non-limiting example a database may include a set of genome sequences of microorganisms known to be present in a specific body organ, for example, the human gut.

A set of different type of compositional spectra is a distribution of imperfect occurrences of random strings in a given text such a polynucleotide.

### Definitions

For convenience certain terms employed in the specification, examples and claims are described herein.

It is to be noted that, as used herein, the singular forms “a”, “an” and “the” include plural forms unless the content clearly dictates otherwise.

Where aspects or embodiments of the invention are described in terms of Markush groups or other grouping of alternatives, those skilled in the art will recognize that the invention is also thereby described in terms of any individual member or subgroup of members of the group.

DNA and Deoxyribonucleic acid are used synonymously to refer to a long chain polymer which comprises the genetic material of most living organisms. The repeating units in DNA polymers are four different nucleotides, each of which comprises one of the four bases, adenine, cytosine, guanine and thymine bound to a deoxyribose sugar to which a phosphate group is attached. Triplets of nucleotides, referred to as codons, in DNA code for amino acids in a polypeptide.

Nucleotide includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in the polynucleotide.

A polynucleotide is nucleic acid sequence of any length and includes oligonucleotides and also gene sequences found in chromosomes.

An oligonucleotide refers to a linear polynucleotide sequence of up to about 50 nucleotide bases in length, for example a polynucleotide (such as DNA or RNA) which is at least about 4 nucleotides, for example at least 6, 10, 25 or 50 nucleotides long.

Microorganisms include the prokaryotes, namely the bacteria and archaea; and various forms of eukaryotes, including protozoa, fungi and algae. Viruses are included in the definition of microorganism, as used herein. Each microorganism has a unique genome, which allows precise identification of its strain and species.

A metagenome refers to a mixture of microorganism genomes. There are three possible situations for a metagenome in a sample:
1) All genomes in the mixture are genomes of known microorganisms. In this case, the solution accuracy depends on the accuracy of the sequenator employed. If the sequenator provides accurate data, the solution is accurate.
2) Some genomes in the mixture are known, while the others are unknown. In this case, it is possible to evaluate only the quantities of known genomes, and there is some error, which depends on the fraction of the unknown genomes in the mixture.
3) All genomes in the mixture are unknown, and for which the method disclosed herein is not applicable.

A mixture set as used herein refers to the genomes making up the metagenome in a sample.

A separating set as used herein refers of a data set of the sequences of known genomes of microorganisms, the set being available, for example, in a public or private database.

The term “purified” does not require absolute purity; rather, it is intended as a relative term. For example, a purified nucleic acid preparation is one in which the subject polynucleotide in the preparation represents at least 25%, at least 50%, or for example at least 70%, of the total content of the preparation. Methods for purification of polynucleotides are well known in the art.

A “sample” refers to a material to be analyzed for example for the presence and composition of microbial genomes. A sample includes a biological sample, an environmental sample, a food sample, a pharmaceutical sample a cosmetic sample and the like. A biological sample includes for example, sputum, vaginal secretion, fecal matter, saliva, blood, a biopsy, cerebrospinal fluid (CSF) pus, synovial fluid]. Biological samples can be obtained for example, in a clinical setting. An environmental sample includes for example soil, plant material and water. Environmental samples can be obtained from an industrial source, a farm and a stream or other water source.

A “sequenator” or “sequencer” refers to an apparatus for determining the order of monomers in a biological polymer, i.e. the order of the nucleosides A, C, G and T in a DNA polynucleotide.

Bacteria include pathogenic bacteria causing infections such as tetanus, typhoid fever, diphtheria, syphilis, cholera, food borne illness, leprosy, peptic ulcer disease, bacterial meningitis, and tuberculosis. Some species of bacteria are part of the natural human flora and yet are able to cause multiple infections in human hosts. For example, Staphylococcus or Streptococcus, can cause skin infections, pneumonia, meningitis and sepsis. Some species including Rickettsia, and Chlamydia are intracellular parasites while other species such as Pseudomonas aeruginosa, and Mycobacterium avium are opportunistic pathogens and cause disease primarily in immunosuppressed individuals.

Viruses include human pathogens, animal pathogens and plant pathogens. Non-limiting examples of viruses include influenza viruses and all of its strains, HIV, hepatitis A, B and C, Epstein-Barr virus, papillomaviruses, herpesvirus, adenovirus, Ebola and SARS.

Non-limiting examples of protozoa include human parasites, causing diseases including malaria, amoebiasis, giardiasis, toxoplasmosis, trichomoniasis, Chagas disease, leishmaniasis, sleeping sickness and dysentery.

The invention has been described in an illustrative manner, and it is to be understood that the terminology used is intended to be in the nature of words of description rather than of limitation.

Many modifications and variations are possible in light of the above teachings. It is therefore, to be understood that within the scope of the appended claims, the invention can be practiced otherwise than as specifically described.

Throughout this application, various publications, including United States Patents, are referenced by author and year and patents by number. The disclosures of these publications and patents and patent applications in their entireties are hereby incorporated by reference into this application in order to more fully describe the state of the art to which this invention pertains.

The present invention is illustrated in detail below with reference to examples, but is not to be construed as being limited thereto.

Citation of any document herein is not intended as an admission that such document is pertinent prior art, or considered material to the patentability of any claim of the present invention. Any statement as to content or a date of any document is based on the information available to applicant at the time of filing and does not constitute an admission as to the correctness of such a statement. Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### EXAMPLES

### Example 1

### Compositional Spectra Analysis

The compositional spectra (CS) of the bacteria in the test samples were calculated based on all possible 6-letter words of the 4 DNA nucleotides (A, C, G, T). Therefore, the CS vector dimension is 4096 and the value of each coordinate is the total number of the corresponding 6-letter word in the genome sequence regarded in both directions ((3′→5′ or 5′→3′).

Calculation Methods. The evaluation of matrix degeneration and conditionality as well as the solution of linear equation systems was performed using the MatLab standard functions. (Kirzhner and Volkovich, March 2012, Evaluation of the Genome Mixture Contents by Means of the Compositional Spectra Method, arXiv:1203.2178v1).

### The Basic Model

Set S={s₁, s₂, . . . , sₘ} of the spectra of m different genomes is considered as a set of vectors in linear space R^{N}, where N is the dimension of the space, which, by definition, equals the number of words in the vocabulary. Greek letter sigma σ=x₁s₁+x₂s₂+ . . . +xₘsₘ is an arbitrary linear combination of these vectors with nonnegative integer coefficients, x. The vector σ is the mixture of the genome spectra s₁, s₂, . . . , sₘ, with coefficients x being the multiplicity of each genome occurrence in the mixture. The problem of mixture separation can be formulated as finding these coefficients for given vectors s₁, s₂, . . . , sₘ and vector σ. If the columns of matrix S are the vectors of set S, the problem is reduced to solving the linear equation (1):

Sx=σ (1)

where matrix S is, generally speaking, a rectangular N×m matrix (N>m) and x is the vector of variables x of dimension m. If matrix S is not degenerate, i.e., vectors s₁, s₂, . . . , sₘ are linearly independent, the linear system has a single solution. Under this condition, there exists a system of vectors T={t₁, t₂, . . . , tₘ} which is bi-orthogonal to the system of vectors S, which, for a standard scalar product, means that the following equalities are true: (tᵢsⱼ)=0 (i≠j) and (tᵢsⱼ)=1 (i=j). Then, (σ,tᵢ)=xᵢ for any i=1, 2, . . . , m.

T is a matrix whose rows are the vectors of set T and the solution of (1) can be written in as equation (2):

x=Tσ (2)

This formula is the solution of the mixture separation problem for the case of a non-degenerate matrix.

The method provided herein for solving the system of equations yields positive or negative coefficients. Small negative coefficients appear as a result of the data noise, while relatively large negative coefficients are indicative of the presence of an unknown genome in the mixture. Therefore, the “direct solution” of the system of equations used herein better reveals the peculiarities of the noise effect than the methods described in the art, thereby providing an advantage over the known methods.

In the model described above, the same genome set is used both for making up the mixture and for building the matrix S. In reality and what follows, these may be, two different genome sets, which are referred to as the mixture set and the separating set, respectively.

### Possible Scenarios and Interpretation of the Solution

If equation (1) is consistent (condition of the model), the problem of arriving at a solution arises when matrix S is degenerate or erroneous. In the latter case, errors in the input data will skew the solution far from reality. Hereinbelow, the two possibilities are considered taking into account the data origin.

The methods known in the art do not take into consideration the following two scenarios a) a degenerate matrix S and b) an erroneous matrix S. These scenarios are biologically relevant and can be interpreted correctly.

a) A degenerate matrix S has a clear biological meaning and the results can be interpreted appropriately. Meinicke (op cit.), asserts that if the number of genomes under consideration, m, is less than the space dimension, N (m<N), there are no biologically significant reasons for the CS vector of one genome to be in the linear span with the CS vectors of the set of other genomes. A random occurrence of such a vector in this linear span also has a zero probability since the volume of the linear span has a zero measure unless it coincides with the entire space.

However, there is an important exception to the rule formulated above and the exception is associated with a biological condition. Two vectors may be considered collinear if both genomes belong to strains of the same species. The two vectors are, actually, more than collinear and are almost equal to each other since such two genomes have, by definition, only minor differences.

Thus, if N>m, it can be supposed that, as a rule, the genome spectra constitute a set of linearly-independent vectors; the only reason for the vectors to be linearly dependent is the coincidence of some of them. In the latter case, the matrix of equation (1) is degenerate as a result of the pair-wise collinearity of some of its columns. For this type of matrix S degeneration, the following method is used to solve the problem: reduce matrix S to S′, arbitrarily leaving one column in each group of pair-wise collinear ones. Then, if system Sx=σ is resolvable, equation S′x=σ has a unique solution, which can be represented using the bi-orthogonal vector set T (as for equation (2)). Namely, if column Sᵢ of matrix S′ had no collinear analogs in matrix S, the value of xᵢ=(σ,tᵢ) is, equal to the multiplicity of vector Sᵢ occurrence in sum σ.

In contrast, if column Sᵢ of matrix S′ had p collinear analogs in matrix S, then equation (3) is relevant:

xᵢ=(σ,tᵢ)=(C₁ᵢx₁+ . . . +Cₚᵢx_{p,} (3)

where the values of xᵢ, . . . xₚ are the multiplicities of the corresponding collinear vector occurrences in sum σ, while coefficients Cⱼᵢ depend on the proportion of vector Sᵢ and its j-th collinear analog lengths and can be calculated a priori. Furthermore, p equations of type (3) can be obtained by choosing, in turn, each of the columns of matrix S as a unique representative of the corresponding group of pair-wise collinear columns. Clearly, the solution of the system of equations (4)

(4)

$${x}_{1} = {C}_{11} {x}_{1} + … + {C}_{p 1} {x}_{p}$$

$$…$$

$${x}_{p} = {C}_{1 p} {x}_{1} + … + {C}_{pp} {x}_{p}$$

allows the unambiguous evaluation of the sums of the occurrence of equal-length genomes in the metagenome. This result suggests that the method does not permit discriminating between bacteria having almost identical genomes, e.g., different strains of a bacterial species and this fact has a clear physical meaning.

b) Conditionality of Matrix S. Bad conditionality of a matrix results from the “almost linear dependence” of its columns. In this case, the system of equations has a unique solution, but its evaluation may be difficult. An “almost linear dependence” is accounted for by the vectors, which are referred to herein as “almost collinear vectors”. Such CS vectors may appear in genome pairs for some biologically significant reasons, e.g., in the case of evolutionary proximity or, alternatively, co-evolution. However, similar to the collinear vectors considered above in (a), almost collinear vectors still require the genomes to be relatively close, which, in turn, suggests that the spectra lengths are approximately equal. The theory, in this case, is almost the same as the theory for the degeneration case, described above. Namely, it can be shown that the solution coordinates, which correspond to the vectors lacking almost collinear analogs, are stable for data fluctuations, while the coordinates corresponding to almost collinear vectors may depend significantly on the data error. Nevertheless, as before, the sums of the coordinates over the whole group of such vectors are stable for data fluctuations.

If the matrix conditionality is so high that it affects precision of the solution, “almost collinear vectors” may be selected and dealt with in the same way as described above for the collinear vectors. Namely, to build a system of bi-orthogonal vectors, only one vector of each pair (group) can be used. This will cause the decrease of the conditionality and the obtained occurrence coefficient will be the sum of the multiplicities of all the bacteria of this group. The solution will include an error, however, the smaller the angle between the “almost collinear vectors”, the smaller the error.

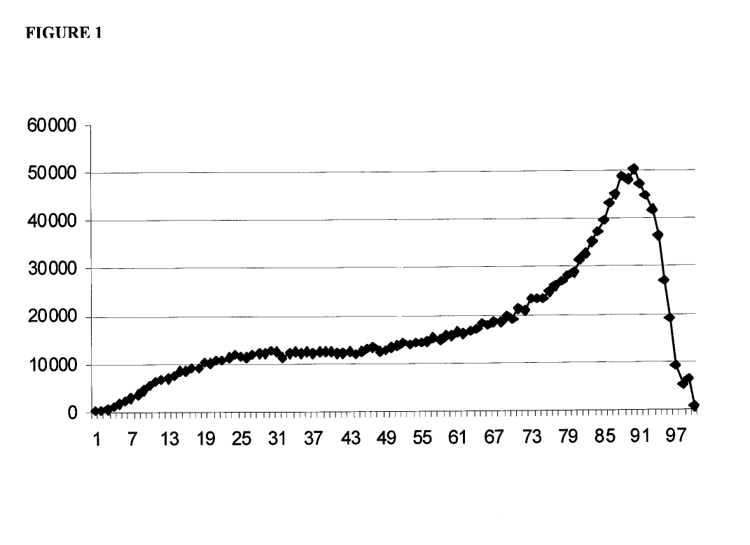

In conclusion, when the genomes of the mixture set and of the separating set are a given, it is possible to a priori obtain the characteristics of matrix S, in particular, its rank and conditionality. Calculating the pairwise scalar products of the vectors of a given set S, it is possible to obtain information on their collinearity and a priori develop an adequate scheme of solution and assess the result. In particular, it is possible to conduct simulations in order to evaluate the level of the solution error. As an example, FIG. 1 demonstrates the distribution of the cosine values for the angles between all possible CS (compositional spectra) pairs for approximately 1300 bacterial genomes. Non-limiting examples of bacterial genome sequences are obtained at the following website http://www.ncbi.nlm.nih.gov/genomes/lproks.cgi

The data presented in FIG. 1, shows that the number of “almost collinear” vectors is relatively small. The corresponding matrix composed of CS for all considered genomes is not degenerate, so, indeed, the genome compositional spectra do not belong to the subspaces generated by the CS of other genome sets. The conditionality of this matrix equals 545. The contribution of vector pairs with high degree of collinearity to this value can be estimated by calculating the conditionalities of the matrixes in which the collinear vectors pairs are eliminated. For example, eliminating one vector in each pair with the cosine values higher than 0.95, 0.98, or 0.99, three matrixes with conditionality values of 74, 199, or 228, respectively, were obtained. Thus, the conditionality values appear to be so high requiring checking the solution accuracy; on the other hand, they are quite compatible with the possibility to solve the problem.

### Results and Discussion

### Testing the Basic Model and Separation of the Mixture in the Absence of Randomness

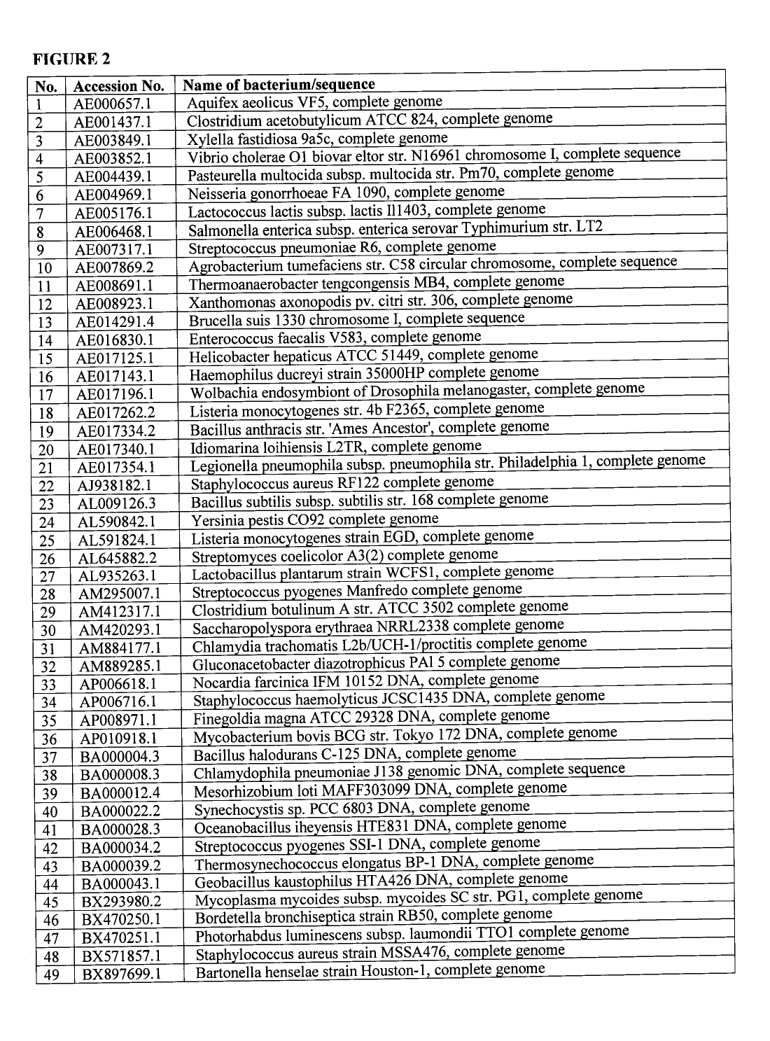

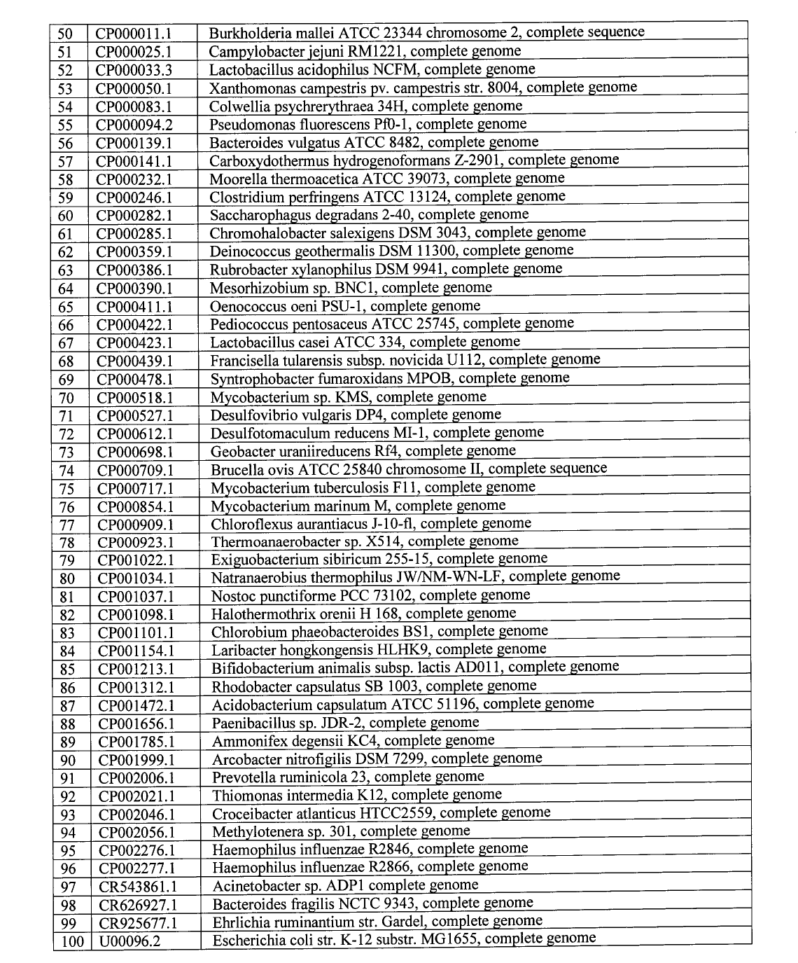

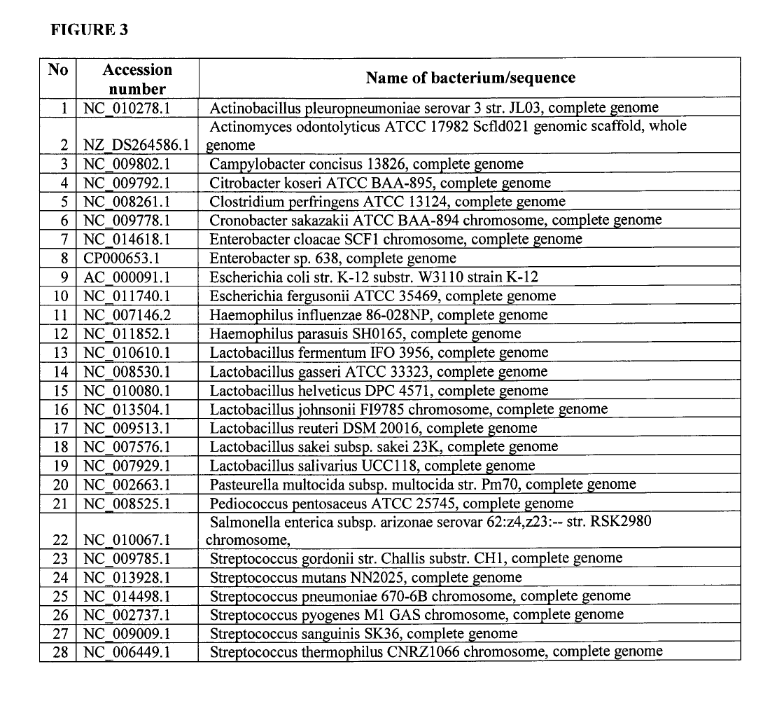

The Genomic Base. To illustrate the calculations in the framework of the described-above model, two sets of genomes were considered. One of the sets, M₁₀₀, contains 100 genomes of Eubacteria, which represents all the main bacterial groups, the number of genomes in each group being approximately proportional to the number of the sequenced genomes in each group. The choice of genomes from each group is random (FIG. 2). The other set, M₁₀₀, consists of 28 bacteria, which have been characterized as the most common gut bacteria (Qin et al., Nature 464 (2010) 59-65) and, have been completely sequenced (FIG. 3).

For CS calculations all possible 6-letter words were used, so that the dimension of the full CS space is equal to 4096 (N=4096). In this way (as shown in Section 1) matrices M₁₀₀ and M₂₈ were created, their dimensions being 100 and 28, respectively.

The Mixture Model. It is supposed that each genome that is present in the mixture is cut into non-overlapping segments of equal length and that the mixture is composed of such segments. The spectrum of a genome mixture is defined as the sum of the spectra of all segments. Mixtures composed of segments of length C=10, 20, 30, 40, 50, 100, 200, 500, 1000, 10000 bp and also, for the sake of comparison, a mixture that consists of whole genomes have been considered. The multiplicities of the genome occurrences in the mixture are chosen randomly in the range of 0-10, once for all the numerical experiments described herein.

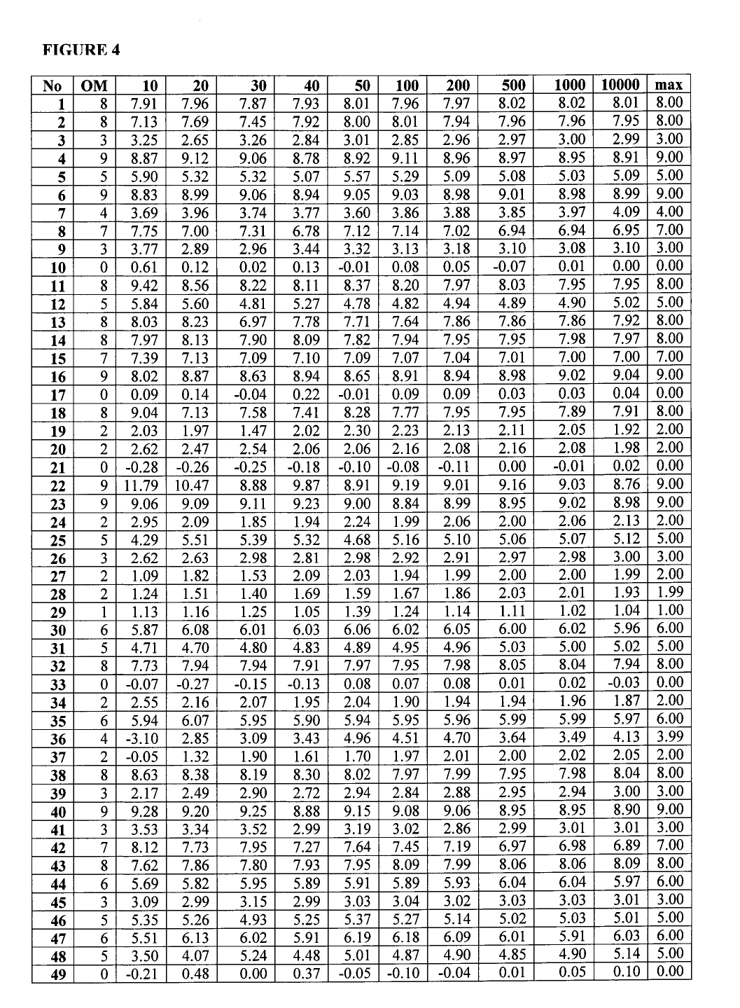

Direct Calculation of Multiplicity. The calculations show that both matrices S₁₀₀ and S₂₈ are non-degenerate. The conditionality of matrices S₁₀₀ and S₂₈ are equal to 314.05 and 78, respectively. However, the relatively high conditionality of matrix S₁₀₀ does not interfere with the possibility of obtaining an almost exact solution of the corresponding system of linear equations in the absence of noise that is not related to the natural computational errors. For example, if a segment is equal to a whole genome (i.e., the mixture spectrum is calculated accurately), the mean deviation from the actual multiplicity value is 0.00179. FIG. 4 presents the results of the calculations of the genome multiplicities in the mixture for different segment lengths and FIG. 5A shows the mean differences between the calculated and the actual genome multiplicities in the mixture.

As explained above, the linear combinations of spectra do not create new spectra, so the poor conditionality of matrix S₁₀₀ may result from the “almost collinearity” of some spectra. The latter suggestion was checked by calculating the cosines of the angles between the vectors (FIG. 5). Although most of the coefficients are not close to 1, a few coefficients were close to 1.

From the data presented in the Table 1, herein below, it can be seen that if almost collinear vectors are eliminated, matrix M100 becomes much more stable. For example, the elimination of 6 genomes results in approximately a 10-fold decrease of the conditionality.

TABLE 1

| | | | Cosine of | Genome | Genome | |
|---|---|---|---|---|---|---|
| #* | Bacteria 1 | Bacteria 2 | angles | 1 length | 2 length | Cond** |
| 36, 75 | Mycobacterium | M. tuberculosis F11 | 0.99991 | 4345 | 4424 | 288 |
| | bovis | | | | | |
| 28, 42 | S. pyogenes | S. pyogenes SSI-1 | 0.998939 | 1841 | 1894 | 285 |
| 95, 96 | H. influenzae R2846 | H. influenzae R2866 | 0.998936 | 1819 | 1932 | 283 |
| 18, 25 | L. monocytogenes | L. monocytogenes | 0.998768 | 2905 | 2944 | 281 |
| | str. 4b F2365 | strain EGD | | | | |
| 22, 48 | S. aureus RF122 | S. aureus | 0.998579 | 2742 | 2799 | 158 |
| | | strain MSSA476 | | | | |
| 12, 53 | X. axonopodis | X. campestris | 0.995408 | 5175 | 5148 | 157 |

*numbers from table in FIG. 2
**conditionality of matrix S100 calculated after the bolded genomes (column 1) have been eliminated

Table 1 shows the most collinear bacteria pairs from set M₁₀₀, arranged in descending order with respect to the collinearity value. Cosines of the angles refers to cosines between the vectors; Cond refers to conditionality of matrix S₁₀₀ calculated after the genomes marked in bold in each row have been eliminated from the entire set M₁₀₀. For example, for the 1^{st} row, the conditionality is calculated for set M₁₀₀ without genome number 75; for the 2^{nd} row, the conditionality is calculated for set M₁₀₀ without genomes number 75 and 28.

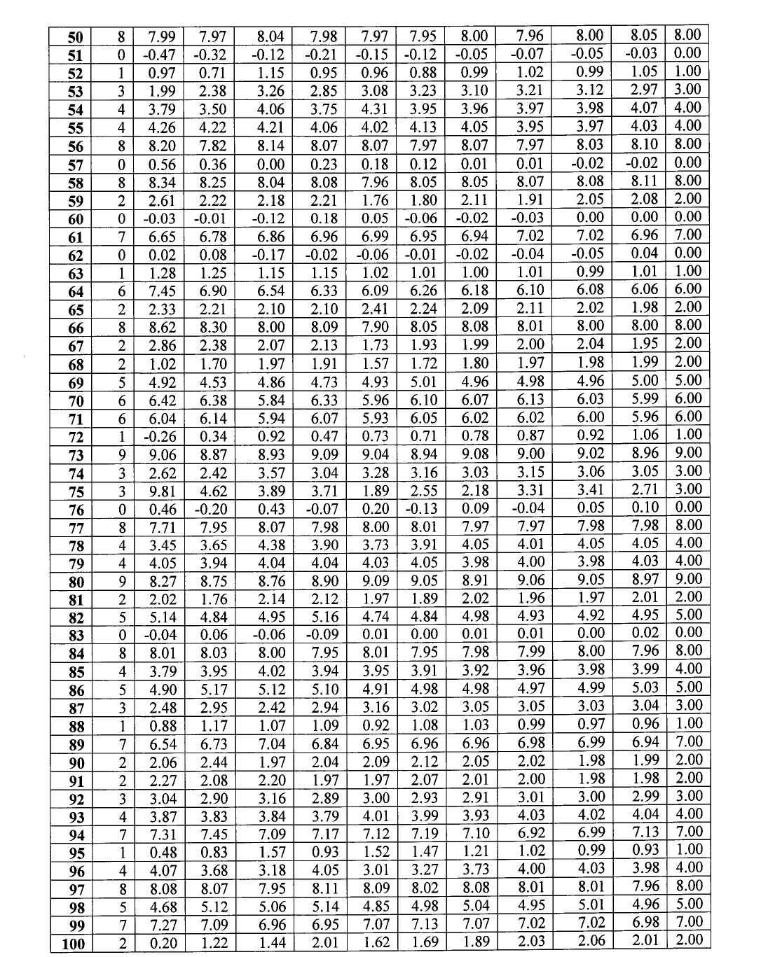

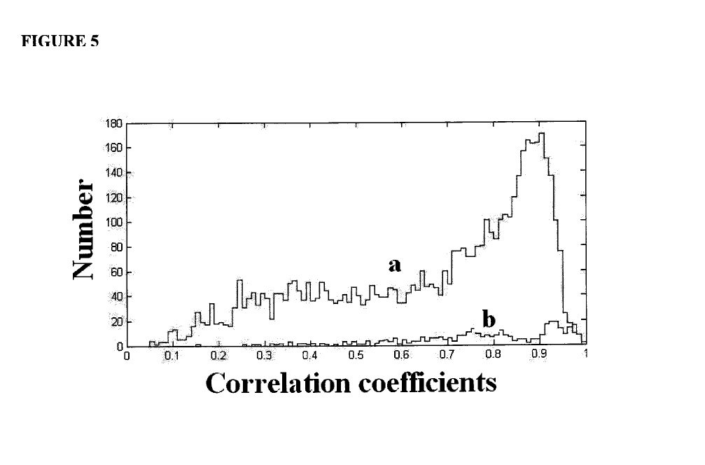

Since the M₂₈ genome set conditionality is good enough for performing calculations, it can be supposed that the angle between the vectors in the almost collinear genome pairs is much larger in this case. Indeed, only for one genome pair (E. coli-E. fergusonii), the cosine value is 0.993 and there are only two other values slightly exceeding 0.98. With the M₂₈ set as both the separating and the mixture set, the calculated mean deviation of the obtained multiplicity from the actual one is 0.04097 if the segment length in the mixture is equal to the genome length. The calculated genome multiplicities for different segment lengths are presented in the table in FIG. 6, while FIG. 7 shows the mean differences between the calculated and the actual genome multiplicities in the mixture.

Reduction of the Separating Set. Another calculation method, which consists of eliminating one vector from each pair of almost collinear vectors of set (those bolded in the first column in the table in FIG. 5B) was employed. The remaining 94 genomes constitute a separating set S₉₄. Employing this set, the multiplicities of the occurrences in the mixture of both genomes (the remaining and the eliminated ones) of the almost collinear pair cannot be calculated separately. The calculated multiplicity of the remaining genome of each almost collinear genome pair is equal to the sum of the multiplicities of the genome itself and the genome lacking from this pair. For example, consider the pair of almost collinear M. Bovis and M. tuberculosis genomes (first set in Table in FIG. 5B). Elimination of the latter genome from the separating set results in the M. Bovis multiplicities equal to 7.2417, 7.9169, and 7.3478 with the segment lengths of 10, 20 and 30, respectively, while the actual summarized multiplicity is equal to 7. The mean difference between the calculated and the actual genome multiplicities in the mixture is shown in FIG. 7.

Noise Effect. Next, in order to demonstrate the effect of matrix S₁₀₀ bad conditionality on the errors in calculating the multiplicities, the calculations for the noise introduced into the mixture vector were performed. Into each coordinate of the accurate spectra, noise was introduced, which was randomly and evenly distributed between 0% and 1% of the coordinate value. As a result, the calculated multiplicity values for the most collinear genome pair, M. bovis-M. tuberculosis (Table 1, above), are 7.14 and 0.03 as compared to the actual values of 4 and 3, respectively. However, the sums of the calculated (7.17) and the actual (7.0) multiplicities are much closer to each other, in accordance with the above considerations. The next two pairs of almost collinear genomes in FIG. 5B are also subject to the introduced error (Table 2, hereinbelow).

TABLE 2

| 1 | 2 | 3 | 4 |
|---|---|---|---|
| 28 | 2 | 1.9944 | 1.639 |
| 42 | 7 | 7.003 | 7.225 |
| sum | 9 | 8.9944 | 8.864 |
| 95 | 1 | 1.0005 | 0.443 |
| 96 | 4 | 4.0012 | 4.539 |
| sum | 5 | 5.0017 | 4.982 |

Table 2. The values of multiplicities calculated in the absence and in the presence of noise as well as the actual values for both pairs. In the header row: 1 represents genome numbers; 2 represents actual multiplicity values and their sums; 3 represents calculated multiplicity values in the absence of noise; 4 represents calculated multiplicity values in the presence of noise.

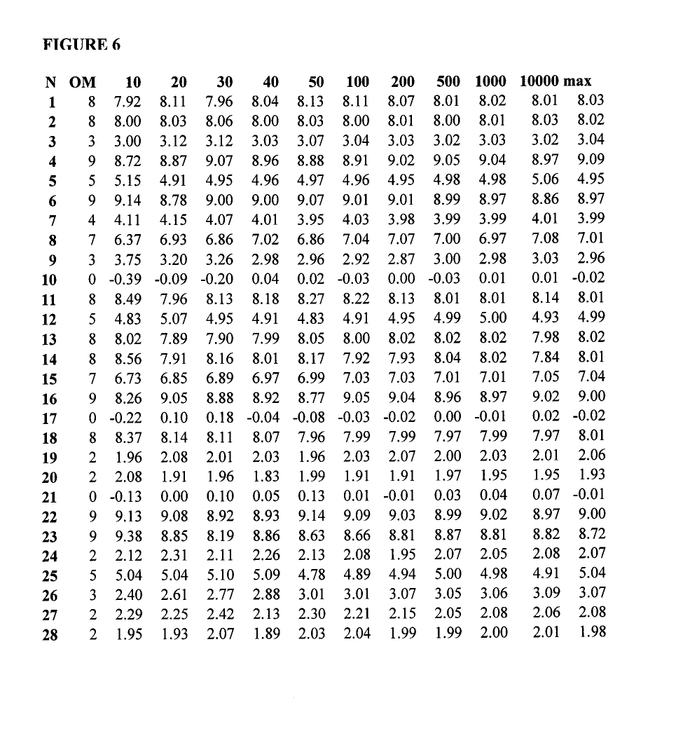

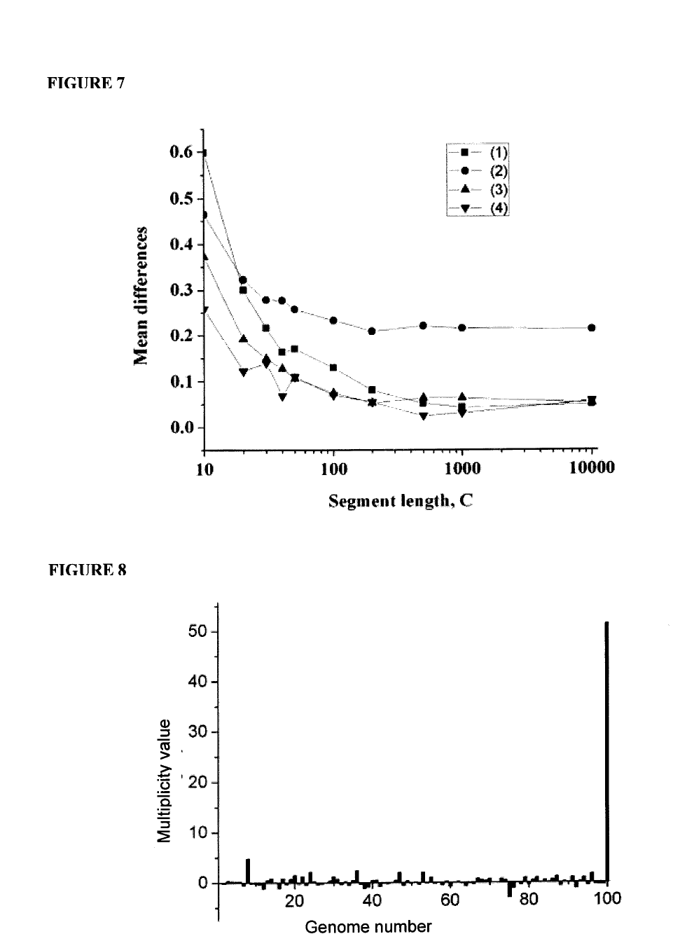

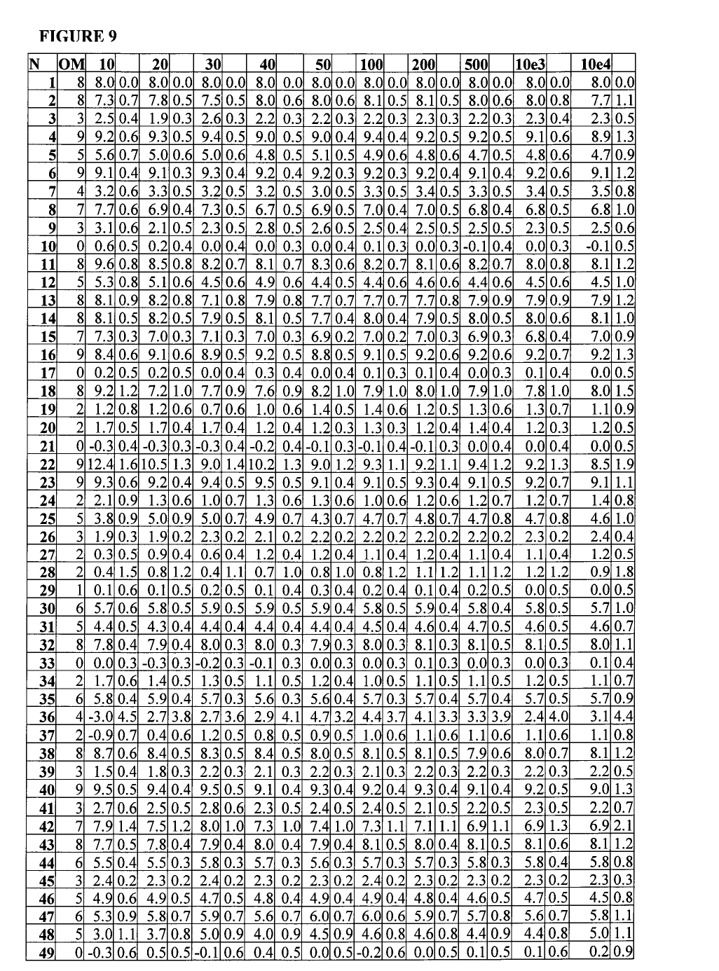

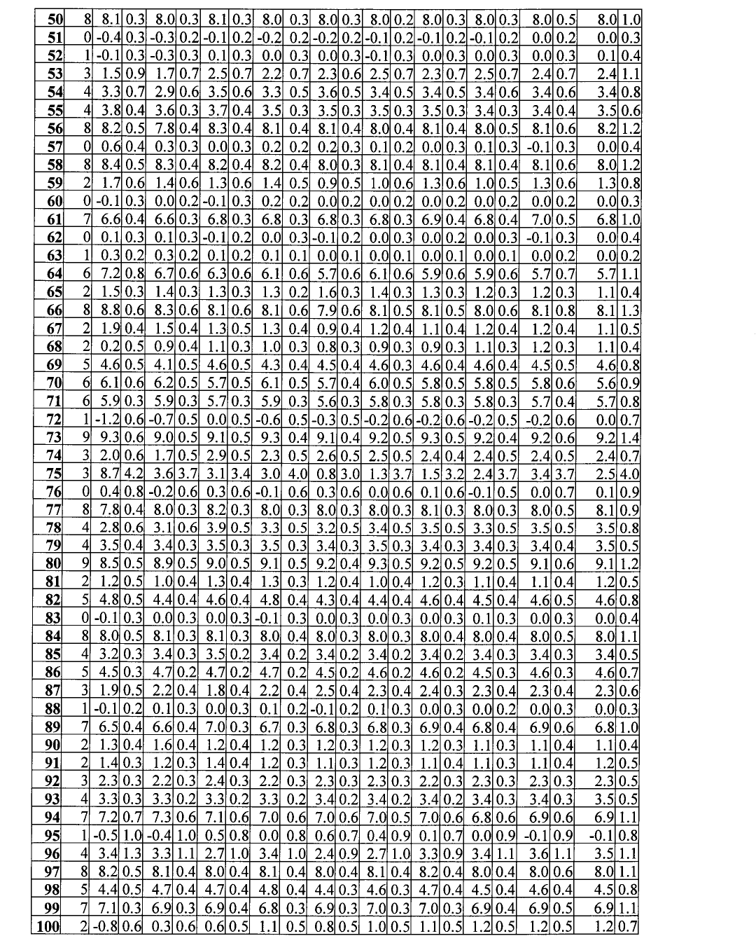

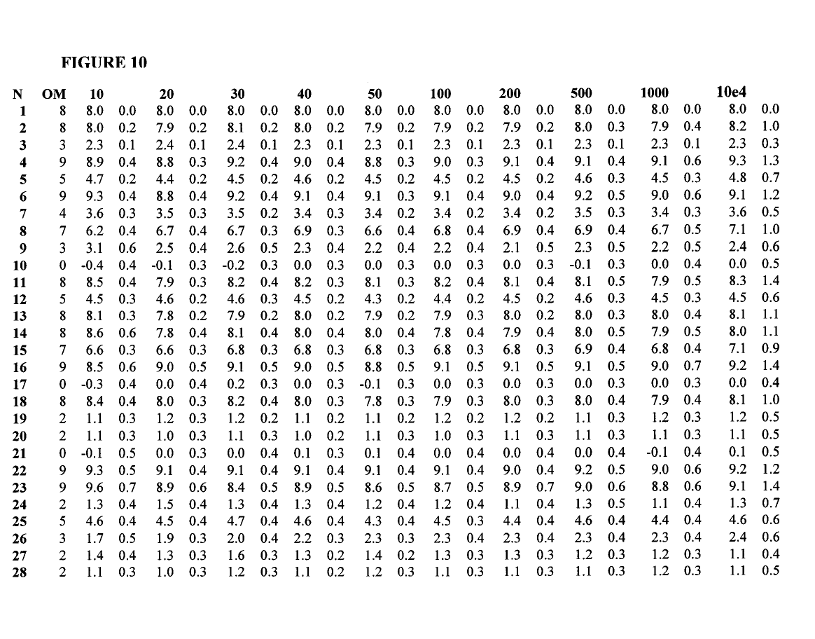

Separating and Mixture Sets are Different. Consider set M₁₁, consisting of 11 different E. coli genomes. The correlation coefficient between each pair of these genomes is larger than 0.99. Let this set be the mixture set and the separating set be set M₁₀₀, which contains only one E. coli genome. The separation obtained for the mixture of the whole genome spectra is presented in FIG. 8.

The calculated total coefficient for the E. coli genome is 50, while the actual one is 64. The other coefficients are not equal to zero, but almost all of them are less than 1 (see FIG. 8). The largest coefficient, equal to 4, corresponds to Salmonella (number 8 in FIG. 2 table), which can be readily understood from the biological point of view, i.e. the genomes of these two bacteria are quite similar, thereby explaining the results obtained.

Consideration of more examples of this issue, i.e., the sets that consist of 200, 500, or 1000 genomes, can hardly clarify the situation any further. It can be expected that with the increase of the genome number, the probability of the occurrence of collinear and almost collinear pairs also increases, which, in turn, increases the conditionality of the system. At the same time, all of the above collinearity possibilities can be tested directly since the properties of known genomes were tested.

### Separation of a Mixture with Random Fluctuations

The following simple model for random generation of a metagenome spectrum will be used.

Model of metagenome random fluctuation and normalization of the result. Consider again genome sets M₁₀₀ and M₂₈. The same integer coefficients x, are used, but the genome spectrum is calculated in a different way. Namely, each genome segment is included in the mixture with an integer value of multiplicity, distributed evenly from 0 to the fixed value x for this genome. The idea of this model is that, actually, not all the segments, but only some random portion of them, are present in the sequenced metagenome. For both sets M₁₀₀ and M₂₈, the model simulation was conducted 100 times for the same segment lengths that were used before.

In contrast to the deterministic case considered above, in the framework of this probabilistic model, the solution of Eq. 1 fundamentally cannot give even the approximate actual multiplicity of a genome in the mixture. The reason for this is that the described procedure efficiently decreases this multiplicity to the level which is determined by the properties of the randomizing process. Although pair-wise multiplicity ratios are preserved, the calculated absolute values must be lower than the actual ones. Assuming different properties of the process of selecting the mixture segments, it is possible to introduce different recovery coefficients. However, a simple technique of normalizing the result, which lies a little bit away from pure theory is proposed herein. Namely, prior to metagenome sequencing, a known number of one or two bacterial species were added to the metagenome. It is desirable that these bacteria be, in biological terms, as far as possible from the supposed composition of the metagenome. Then the ratio of the known multiplicity of each of these bacteria to the calculated multiplicity will be the sought for proportion coefficient for all the bacteria in the mixture. In the following computer experiments, the first genome on the list was considered to be such an added genome. The same method can be successfully used in the estimation of the inaccuracy caused by the ill-conditionality of the system.

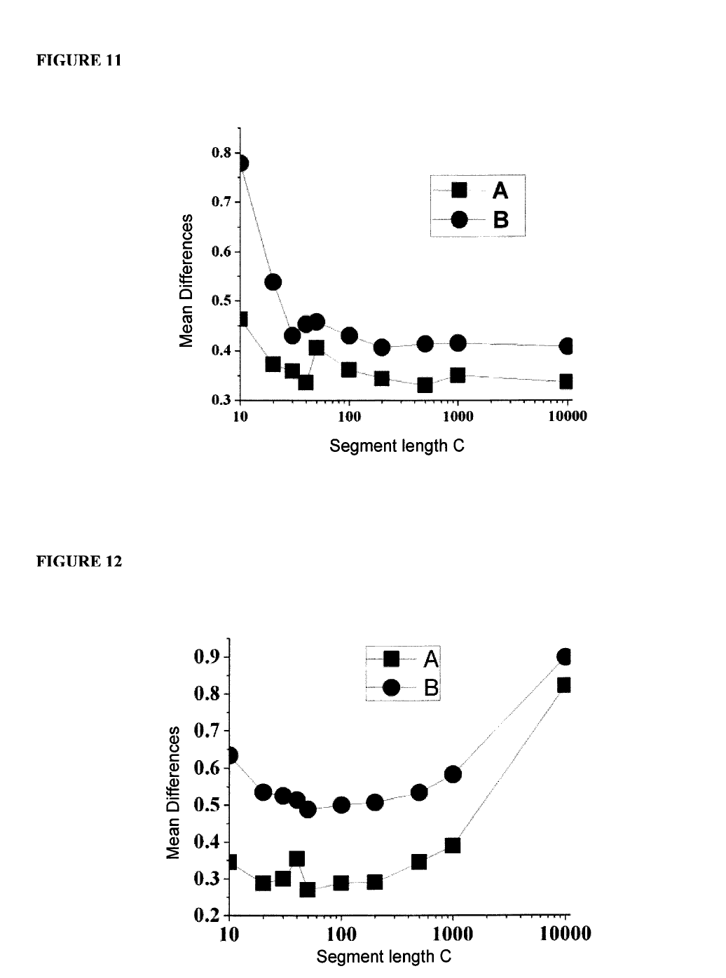

Experiments with the Fluctuation Model. The characteristics calculated in this case were the mean multiplicity value dᵢ (i=1, . . . , 100) for each bacterium and the squared deviation σᵢ for each dᵢ (Figures) (averaging was performed over 100 experiments in each series). Calculating deviations dᵢ from the corresponding actual multiplicities and averaging these values over all bacteria, the quality of solving the mixture-separation problem at different segment length values in the mixture was assessed (shown in FIG. 11).

From the data presented in FIG. 11, it can be seen that different segment lengths result in different mean errors, the dependence being non-monotonous. The mean values of the mean-squared deviation are shown in FIG. 12. On the whole, this characteristic increases at the ends of the segment-length ranges.

The curves presented in FIGS. 11 and 12 suggest that the fragments of length 40, 50 bp give better results than large-length fragments provided that the probability of losing a segment does not depend on its length. It should be noted that the results for almost collinear pairs of bacteria are qualitatively the same as already obtained with noise artificially introduced into the mixture vector. The results for the two most collinear pairs from set M₁₀₀ (Table 1) are presented in Table 3, hereinbelow. The actual and calculated multiplicities for each genome from set M₂₈ at C=50 or 10000 are shown in FIG. 7.

TABLE 3

| N | AM | 10 | 20 | 30 | 40 | 50 |
|---|---|---|---|---|---|---|
| 36 | 4 | −3.01 | 2.67 | 3.68 | 2.86 | 4.68 |
| 75 | 3 | 8.74 | 3.58 | 3.11 | 3 | 0.77 |
| sum | 7 | 5.73 | 6.25 | 6.79 | 5.86 | 5.45 |
| 28 | 2 | 0.43 | 0.77 | 0.43 | 0.69 | 0.82 |
| 42 | 7 | 7.92 | 7.52 | 8 | 7.28 | 7.37 |
| Sum | 9 | 8.35 | 8.29 | 8.43 | 7.97 | 8.19 |

Table 3 shows the actual and the calculated multiplicities for two genome pairs in the case of random fluctuations. N represents genome number; AM refers to actual multiplicity, 10, 20, . . . , 50—segment lengths. In the case of the first pair, the actual multiplicity cannot be calculated (−3.01 as compared to 4 and 8.74 as compared to 3). However, the sums of the actual (7) and calculated (5.73) multiplicities are much closer. For all the mixtures, the sum of the obtained multiplicities equals approximately 6. Similarly, for the second pair, the difference between the actual and the calculated multiplicities is much larger than the difference between the corresponding sums (9 for the actual and about 9 for the calculated multiplicities).

Effect of the Separating Set Growth. As shown above, certain violation of the basic model conditions, i.e., the assumption that the mixture genome set may not be a subset of the separating set (system (1) is inconsistent in this case), still allows application the model quite effectively. In the cases analyzed above, the differences between these sets were minimal—the mixture set contained the genomes which did not belong to the separating set, but had almost collinear analogs there. In order to increase the probability of such a situation, it is preferred that the set of all sequenced genomes be chosen as a separating set since the composition of the mixture cannot be influenced. Thus the efficiency of the method increases with an increase in the set of known genomes.

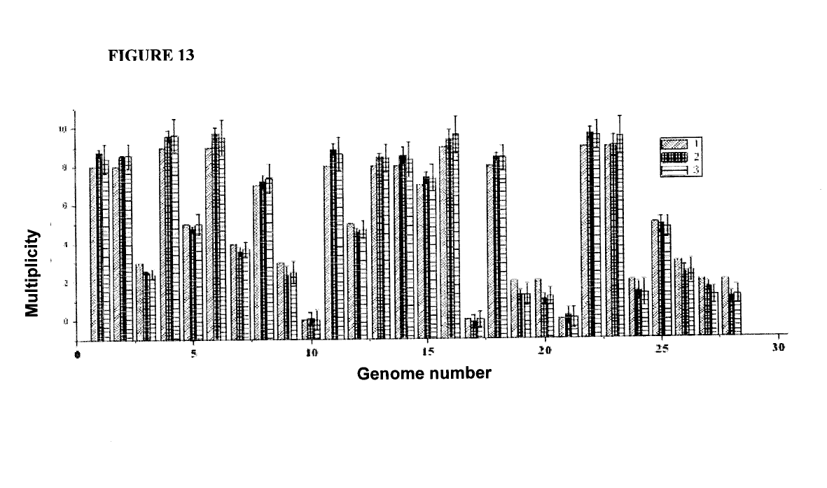

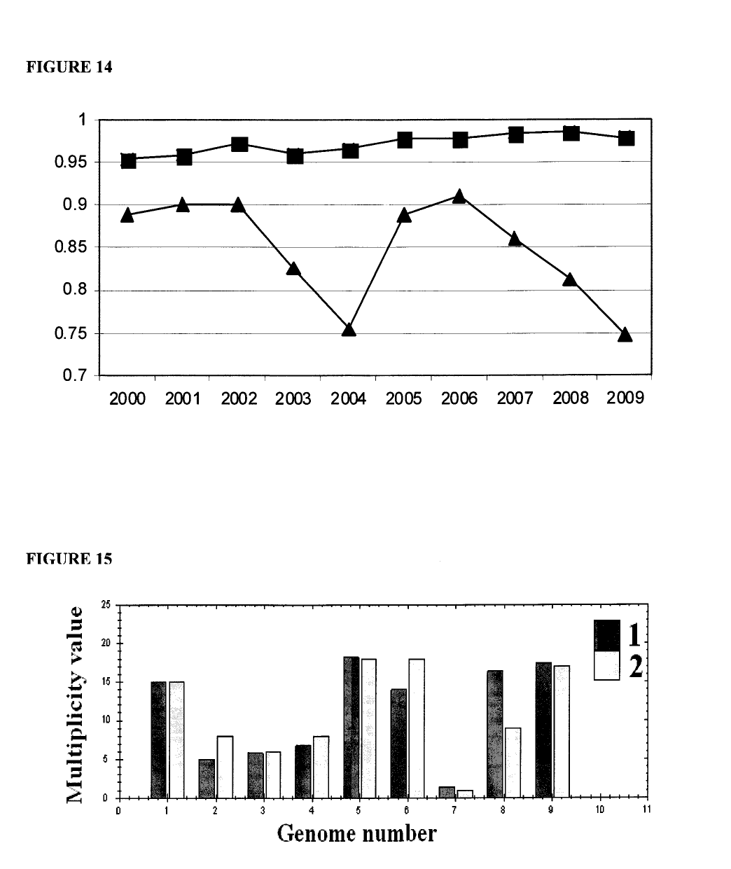

To illustrate this statement, FIG. 14 shows the dynamics of the angles between the new and the known sets of genomes over the last ten years. It can be seen that in this period, these angles have been decreasing although each year, there appeared a genome significantly different from those sequenced before. Nevertheless, sooner or later, the variety of microorganisms will be reduced to the variations of genomes around the forms already studied. In this case, a mixture spectrum can be viewed as a sum of known genomic spectra and the same spectra with some variations. In other words, the spectra of unknown microorganisms will not differ significantly from those of the corresponding known microorganisms. Under these conditions, the multiplicities (coefficients) in the mixture of the known genomes can be obtained using the method described herein based on applying a bi-orthogonal basis or other methods of solving an inconsistent system. As shown above, the calculated multiplicities of genomes in the mixture are related not only to a particular genome, but also to all the other similar genomes, which, however, do not belong to the separating set (and thus are unknown). A plausible biological assumption is that these are unknown genomes which are close to this particular genome and encode similar biological traits. In this way, the qualitative contents of the mixture can be evaluated.

Linear Genome Space. Clearly, the expansion of the genome set requires an increase of the word space. For 6-letter words, the theoretically plausible limit of the space dimension is 4096 and the number of known genomes will soon exceed this value. Actually, the linear dimension of such a set is twice as small due to the existence of special word symmetry—extended Chargaff's second parity rule [Forsdyke et al., Applied Bioinform. (2004) 3:3-8]. This empirical rule, which claims that “reverse-complement” words (e.g. ATTGC<==>GCAAT) almost always have the same occurrence frequency in a genome.

It is possible to work with words of larger length, e.g., 7, 8, or 10 bp. However, the shorter the word chosen for constructing the CS, the shorter each fragment may be in the metagenome to which the present method is applied. Additionally, bacterial genomes are usually of rather limited length and, therefore, relatively long words rarely occur in such genomes. For this reason, their occurrence frequencies become statistically unstable. For example, in a 10⁶ bp-long sequence, words 6, 7, 8, 9, and 10 bp in length occur, on average, 250, 62, 13, 3 times and only once, respectively.

A linear dimension that is generated by the set of 7- or 8-letter words will soon become less than the number of sequenced genomes. However, with regard to the extended Chargaff s rule described above, the linear dimension of the set of all 9-letter words is approximately 100,000. The present method further includes calculating each word's occurrence in the sequence even with one- or two-letter mismatch as described (Kirzhner, et al. (2012) Physica A 312). Thus, along with each word, 351 words close to it (according to the standard evolutionary substitution metrics) also contribute to the total occurrence value. Such number of words ensures statistically significant occurrence values and the method has already proved to be effective, in particular, in the bacteria genome classification problems [Kirzhner, et al., J. Molecular Evolution (2007) 64 (4):448-456; Volkovitch et al., Pattern Recognition (2010) 43 (3):1083-93]. An example of separating a genome mixture using a vocabulary that contains 200 10-letter words, with a three-letter mismatch is shown in FIG. 15. Due to statistical stability, not all possible words of particular length have to be chosen as the basis; the number of such words is less and depends on the volume of the genome set under consideration.

### CONCLUSION

The novel method of genome mixture separation proposed in Meinicke et al. has been tested for separating a mixture that consists only of sequenced genomes. The present method developed and expanded the method of Meinicke and has adapted it for clinical and environmental use by taking into account the large conditionality, which requires estimating the solution quality depending on the data error. The dependence of the solution quality on the fragment lengths in the metagenome, on random errors, etc is described above. Furthermore, in some embodiments the method comprises adding a “neutral” bacterium to the metagenome, allowing estimating the impact of errors of different types on the solution quality to provide a real-life application of the method.

### Example 2

### Biological Software Validation

In view of the intensive pace of current research, all genomes having clinical and environmental relevance will be sequenced in the near future. Therefore, the metagenome content of known microbial genomes will become the norm. Two experiments are conducted to validate the algorithm:

1. Mixed Culture of Bacteria: Culturing of bacteria in vitro. Six to ten different bacterial strains are cultured individually in liquid culture for 24 hours. Subsequently, different volumes are taken from each culture and mixed together to form one culture at predetermined ratios. Aliquots are taken from each overnight culture and spread on a petri dish to determine bacterial number per milliliter. These data are used to determine the ratio of the bacteria in the mixed culture. An aliquot of the mixed culture is sequenced, the sequencing data analyzed using the method disclosed herein, and compared to the actual data.

2. The second validation is performed using blood samples drawn from patients suffering from bacteremia. This retrospective validation is done in collaboration with an infectious disease department of one of the tertiary medical centers in Israel and is headed by an infectious disease specialist. Blood samples from patients suffering from bacteremia are collected at the hospital and sequenced using a DNA sequencer to obtain the corresponding metagenome. As part of the regular treatment at the hospital the same samples are cultured to identify the pathogens in the culture. The first pathogens of interest include: Staphylococcus aureus (non-MRSA and MRSA), Streptococcus pyogenes, Pseudomonas aeruginosa, Clostridium difficile, Vancomycin-resistant enterococcus (VRE) and Tuberculosis. These pathogens were selected based on the need for early identification and the expected benefit from early pathogen driven treatment (e.g. reduction in the use of broad spectrum antibiotics which is one of the main causes of bacterial resistance). Specific primers for these pathogens are used to sequence the bacteria and the sequence results are to be compared with the organisms identified by cultivation and dye-based diagnosis tests.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the removed material is specifically recited herein. Other embodiments are within the following claims.

The invention claimed is:

1. A method for characterizing a microorganism metagenome in a sample, the method comprising
a) providing a compositional spectra mixture from genomic sequences of genomes comprising the microorganism metagenome in the sample;
b) providing a compositional spectra set of known microorganism genomic sequences,
c) characterizing sequences in the compositional spectra mixture of (a) using the compositional spectra set of (b), wherein said characterizing
comprises solving a linear system by (i) providing a vector of representations of said sequences in said compositional spectra mixture of (a); and (ii) comparing representations in said vector to representations of sequences in said compositional spectra set of (b).

2. The method of claim 1, wherein step (c) is performed by a suitably configured processor of a computer system stored on a computer readable medium configured to receive the compositional spectra mixture and the compositional spectra set.

3. The method of claim 1, wherein step (c) is performed by a suitably configured processor of a computer system stored on a computer readable medium comprising the database of known microorganism genomic sequences, and configured to receive the compositional spectra mixture.

4. The method of claim 1, wherein the providing said compositional spectra mixture in step (a) comprises employing a sequenator to provide said compositional spectra mixture.

5. The method of claim 4, wherein the providing the compositional spectra mixture comprises providing fixed length strings of nucleosides (words) based on the genomic sequences.

6. The method of claim 5, wherein the fixed-length string of nucleosides is 4 to 20 nucleotides in length.

7. The method of claim 5, wherein each genome sequence is composed of sequence segments of 10 to 10,000 nucleotides in length.

8. The method of claim 7, wherein each genome sequence is composed of sequence segments of 100 to 1,000 nucleotides in length.

9. The method of claim 1, wherein the metagenome in the sample consists of genome of a single microorganism.

10. The method of claim 1, wherein the metagenome in the sample consists of a plurality of microorganisms.

11. The method of claim 1, wherein the characterizing comprises identifying and quantifying each microorganism genome of the metagenome in the sample.

12. The method of claim 1, further comprising prior to providing the genomic sequence, the addition of a microorganism genome having a known genomic sequence to the sample.

13. The method of claim 12, wherein the microorganism genome is unrelated to the metagenome.

14. The method of claim 1, wherein the sample is selected from the group consisting of a food or beverage sample; a pharmaceutical sample; a human/animal sample and an environmental sample.

15. The method of claim 14, wherein the sample is a human sample selected from the group consisting of stomach contents; intestinal contents; urine; blood, vaginal secretion; fecal matter, phlegm (sputum), cerebrospinal fluid (CSF), pus and synovial fluid.

16. The method of claim 14, wherein the sample is an environmental sample selected from the group consisting of water, plant material and soil.

17. The method of claim 1, wherein each microorganism genome in the metagenome is a bacterium genome.

18. A system comprising at least one processor programmed to perform the method of claim 1.

19. A system for characterizing a metagenome in a sample, the system comprising a computer means configured to:
generate compositional spectra set of known genome sequences;
form a stable system matrix by preprocessing a linear system derived from the compositional spectra;
characterize a compositional spectra mixture of the metagenome of the sample using the stable system matrix to solve the linear system by (i) providing a vector of the compositional spectra mixture of the sample's metagenome; and (ii) comparing the vector values to the compositional spectra set of the known microorganism genomes.

20. A machine-readable storage medium comprising a program containing a set of instructions for causing a system to execute procedures for characterizing the metagenome in the sample, the procedures comprising:
generating a compositional spectra set of known genome sequences;
forming a stable system matrix by preprocessing a linear system derived from the compositional spectra set;
characterizing a compositional spectra mixture of the metagenome of the sample using the stable system matrix to solve the linear system by (i) providing a vector of representations of said sequences in said compositional spectra set; and (ii) comparing representations in said vector to representations of sequences in said compositional spectra mixture.
